(19) United States
(12) Patent Application Publication
(10) Pub. No.: US 2015/0307428 A1
(43) Pub. Date: Oct. 29, 2015

(54) PROCESS FOR PREPARING ALKOXYLATED ALCOHOLS

(71) Applicant: SHELL OIL COMPANY, Houston, TX (US)

(72) Inventors: James Laurel BUECHELE, Houston, TX (US); Hendrik DIRKZWAGER, ¿t Harde (NL); Michiel Barend ELEVELD, Amsterdam (NL)

(21) Appl. No.: 14/649,569

(22) PCT Filed: Dec. 7, 2013
(86) PCT No.: PCT/EP2013/075630

§ 371 Date: Jun. 4, 2015

(30) Foreign Application Priority Data
Dec. 7, 2012 (EP) 12196175.9

(51) Int. Cl. C07C 41/46; C07C 303/24

(52) U.S. Cl. CPC C07C 41/46; C07C 303/24

(57) ABSTRACT

The invention relates to a process for preparing alkoxylated alcohols, wherein an alkoxylated alcohol which contains more than 200 parts per million by weight of a Group IA or Group IIA metal ion is contacted with a sulfonic acid. The resulting alkoxylated alcohol may then be sulfated by contacting it with a sulfating agent.

### FIELD OF THE INVENTION

The present invention relates to a process for preparing alkoxylated alcohols.

### BACKGROUND OF THE INVENTION

Processes for preparing alkoxylated alcohols are well known in the art. Typically, such processes involve the reaction of a starting alcohol having one or more active hydrogen atoms with one or more alkylene oxides, such as ethylene oxide, propylene oxide, butylene oxide or mixtures of two or more of these. Suitable starting alcohols include monofunctional alcohols containing 1 hydroxyl group and polyfunctional alcohols which may contain of from 2 to 6 hydroxyl groups. Examples of said monofunctional alcohols are alcohols of formula R—OH, wherein R is an aliphatic group and the alcohol is primary or secondary, preferably primary. Examples of said polyfunctional alcohols are diethylene glycol, dipropylene glycol, glycerol, pentaerythritol, trimethylolpropane, sorbitol and mannitol.

Usually, a strong base like potassium hydroxide is used as a catalyst in the above-described alkoxylation reaction. It is common to use such catalyst in an amount of from 0.1 to 0.5 wt. % based on total weight of the reaction mixture. In order to prevent the catalyst to cause any side-reaction in any subsequent step and/or discoloration of the final product, it is known to treat the product containing residual catalyst. For example, the catalyst in that product may be precipitated by adding for example a phosphate. The resulting precipitate, for example potassium phosphate, should then be removed by filtration. Further, it is known to subject such reaction mixture containing residual catalyst to extraction, for example washing, and to adsorption using various adsorbants, for example ion exchange media. Even though said methods result in a removal of the catalyst from the final product, they are cumbersome as they comprise multiple steps which involves additional time, equipment expense and/or solvent expense. Alternatively, it is known to add an acid, for example acetic acid, in order to neutralize the remaining catalyst, for example potassium hydroxide. In this way another salt, for example potassium acetate, is formed which could be advantageously left in the alkoxylated alcohol product.

An example of a specific application where the alkoxylated alcohol product may be used is in a process wherein it is sulfated. In such sulfation process, it is important that the salt formed upon reaction of the residual catalyst with an acid in the preceding alkoxylation step, as described above, is dissolved in the alkoxylated alcohol product and does not form a precipitate. Typically, such sulfation process is carried out in a film type reactor, such as a falling film reactor, in which sulfur trioxide gas (sulfating agent) is absorbed in a liquid flowing down along the reactor inner wall. One disadvantage of having precipitates in the alkoxylated alcohol product is that the distribution of the alkoxylated alcohol over such reactor wall becomes suboptimal. Furthermore, the precipitate may adhere to the inner walls of the reactor thereby enabling undesired side-reactions, such as for example “charring”. Said disadvantages are exemplified hereinbefore with reference to a sulfation process, but may be generally applicable to any process wherein alkoxylated alcohol product is further converted into other valuable chemical products.

Therefore, it is an object of the present invention to provide a process for preparing alkoxylated alcohols wherein alkoxylated alcohol product containing residual catalyst is contacted with an acid that does not result in a precipitate or results in less precipitate.

### SUMMARY OF THE INVENTION

Surprisingly it was found that the above object is achieved by contacting the alkoxylated alcohol product containing residual catalyst with a sulfonic acid.

Accordingly, the present invention relates to a process for preparing alkoxylated alcohols, wherein an alkoxylated alcohol which contains more than 200 parts per million by weight of a Group IA or Group IIA metal ion is contacted with a sulfonic acid. Said Group IA or Group IIA metal ion may originate from the alkoxylation catalyst used in a preceding alkoxylation step.

WO199319113 relates to a method of preparing a hydroxy-functional polyether comprising contacting (a) a hydroxy-functional polyether containing less than or equal to 200 ppm of a Group IA or Group IIA metal ion, and (b) an acid. Said Group IA or Group IIA metal ion may be selected from potassium, sodium, barium and mixtures thereof. Further, said acid may be selected from a group of acids which includes sulfonic acids, specifically dodecylbenzene sulfonic acid, naphthalene sulfonic acid, benzene sulfonic acid, toluene sulfonic acid and methane sulfonic acid.

According to WO199319113, it is preferred to pre-treat the polyether to remove excess catalyst. In WO199319113, it is stated: “To simply neutralize such a high level of catalyst may result in formation of a turbid solid/liquid solution, which may in some cases necessitate processing to remove the large amounts of salts produced thereby, particularly when such is necessary to meet solids content specifications.”. In the examples of WO199319113, extractions were indeed carried out to remove excess potassium hydroxide to a level of about 50 ppm before contacting with the acid.

In the present invention, it has surprisingly been found that such pre-treatment as described above is not necessary and that alkoxylated alcohol containing a relatively large amount of a Group IA or Group IIA metal ion, that may originate from an alkoxylation catalyst, can simply be contacted with a sulfonic acid without formation of a precipitate or with the formation of only a small amount of precipitate. As demonstrated in the below Examples, contacting such alkoxylated alcohol with a sulfonic acid resulted in a non-turbid (clear) alkoxylated alcohol containing substantially no solid precipitate, as opposed to other acids which were also tested.

### DETAILED DESCRIPTION OF THE INVENTION

In the present process for preparing alkoxylated alcohols, the alkoxylated alcohol which contains more than 200 parts per million by weight (ppmw) of a Group IA or Group IIA metal ion is contacted with a sulfonic acid. A sulfonic acid is of the general formula (I)

R—S(═O)₂—OH Formula (I)

wherein R is a hydrocarbyl group.

In the present invention, the hydrocarbyl group R in the above formula (I) may be an alkyl group, cycloalkyl group, alkenyl group or aromatic group, suitably an alkyl group or aromatic group, more suitably an aromatic group. Said hydrocarbyl group may be substituted by another hydrocarbyl group as described hereinbefore or by a substituent which contains one or more heteroatoms, such as a hydroxy group or an alkoxy group.

When said hydrocarbyl group R is an alkyl group, said alkyl group may be a linear or branched alkyl group containing a number of carbon atoms within wide ranges, for example of from 1 to 20, suitably 1 to 15 carbon atoms. A suitable example of a sulfonic acid wherein R is an alkyl group is methane sulfonic acid.

When said hydrocarbyl group R is an aromatic group, R is preferably a phenyl group or a group comprising 2 or more phenyl groups which may be fused. Suitable examples of a sulfonic acid wherein R is an aromatic group are benzene sulfonic acid and naphthalene sulfonic acid.

Preferably, the sulfonic acid to be used in the present invention is a compound of the above formula (I) wherein R is a phenyl group which may be substituted or unsubstituted, preferably substituted. Preferably, said phenyl group is substituted by 1 or more, preferably 1, 2 or 3, hydrocarbyl groups as described hereinbefore. Preferably, said phenyl group is substituted by 1 or more, preferably 1, 2 or 3, alkyl groups. Said alkyl substituents may be linear or branched, preferably linear, alkyl groups containing a number of carbon atoms within wide ranges, for example of from 1 to 40, suitably 1 to 30, more suitably 1 to 20, more suitably 5 to 18, more suitably 8 to 16, more suitably 10 to 14, most suitably 10 to 13 carbon atoms. In a case where said alkyl substituent is linear and contains 3 or more carbon atoms, the alkyl substituent is attached either via its terminal carbon atom or an internal carbon atom to the benzene ring, preferably via its internal carbon atom. Preferably, said substituent or at least 1 of said substituents is attached to the para-position of the benzene ring relative to the S(═O)₂—OH group. Suitable examples of a sulfonic acid wherein R is a phenyl group that is alkylated on the para-position, relative to the S(═O)₂—OH group, are para-toluene sulfonic acid and para-dodecylbenzene sulfonic acid. Particularly suitable in the present invention is para-dodecylbenzene sulfonic acid, also referred to as para-lauryl sulfonic acid. Further, particularly suitable in the present invention are para-alkylbenzene sulfonic acids wherein the alkyl group is mostly linear, and wherein the linearity of the alkyl group is preferably greater than 80%, more preferably greater than 90%, most preferably greater than 95%, and wherein the carbon numbers for the alkyl group are distributed over 10, 11, 12 and 13 carbon atoms, for example as follows: 5 to 15% C10, 20 to 40% C11, 20 to 40% C12 and 20 to 40% C13.

In the present invention, the alkoxylated alcohol which contains more than 200 parts per million by weight of a Group IA or Group IIA metal ion that is to be contacted with the above-described sulfonic acid, is of the following formula (II)

R—O—[R′—O]ₓ—H Formula (II)

wherein R is a hydrocarbyl group (originating from the non-alkoxylated alcohol R—OH), R′—O is an alkylene oxide group (originating from the alkylene oxide used in the alkoxylation) and x is the number of alkylene oxide groups R′—O.

In the present invention, the hydrocarbyl group R in the above formula (II) may be aliphatic or aromatic, suitably aliphatic. When said hydrocarbyl group R is aliphatic, it may be an alkyl group, cycloalkyl group or alkenyl group, suitably an alkyl group. Said hydrocarbyl group may be substituted by another hydrocarbyl group as described hereinbefore or by a substituent which contains one or more heteroatoms, such as a hydroxy group or an alkoxy group.

The non-alkoxylated alcohol R—OH, from which the hydrocarbyl group R in the above formula (II) originates, may be an alcohol containing 1 hydroxyl group (mono-alcohol) or an alcohol containing of from 2 to 6 hydroxyl groups (poly-alcohol). Suitable examples of poly-alcohols are diethylene glycol, dipropylene glycol, glycerol, pentaerythritol, trimethylolpropane, sorbitol and mannitol. Preferably, in the present invention, the hydrocarbyl group R in the above formula (II) originates from a non-alkoxylated alcohol R—OH which only contains 1 hydroxyl group (mono-alcohol). Further, said alcohol may be a primary or secondary alcohol, preferably a primary alcohol.

The non-alkoxylated alcohol R—OH, wherein R is an aliphatic group and from which the hydrocarbyl group R in the above formula (II) originates, may comprise a range of different molecules which may differ from one another in terms of carbon number for the aliphatic group R, the aliphatic group R being branched or unbranched, number of branches for the aliphatic group R, and molecular weight.

Preferably, the hydrocarbyl group R in the above formula (II) is an alkyl group. Said alkyl group may be linear or branched, and contains a number of carbon atoms within wide ranges, such as from 5 to 30, suitably 5 to 25, more suitably 10 to 20, more suitably 11 to 19, most suitably 12 to 18. In a case where said alkyl substituent is linear and contains 3 or more carbon atoms, the alkyl substituent is attached either via its terminal carbon atom or an internal carbon atom to the oxygen atom, preferably via its terminal carbon atom.

The alkylene oxide groups R′—O in the above formula (II) may comprise any alkylene oxide groups. For example, said alkylene oxide groups may comprise ethylene oxide groups, propylene oxide groups and butylene oxide groups or a mixture thereof, such as a mixture of ethylene oxide and propylene oxide groups. In case of a mixture of ethylene oxide and propylene oxide groups, the mixture may be random or blockwise. Preferably, said alkylene oxide groups consist of propylene oxide groups.

In the above formula (II), x represents the number of alkylene oxide groups R′—O. In the present invention, the average value for x may be at least 0.5, suitably of from 1 to 25, more suitably of from 2 to 20, more suitably of from 3 to 18, most suitably of from 4 to 16.

The non-alkoxylated alcohol R—OH, from which the hydrocarbyl group R in the above formula (II) originates, may be prepared in any way. For example, a primary aliphatic alcohol may be prepared by hydroformylation of a branched olefin. Preparations of branched olefins are described in U.S. Pat. No. 5,510,306, U.S. Pat. No. 5,648,584 and U.S. Pat. No. 5,648,585, the disclosures of all of which are incorporated herein by reference. Preparations of branched long chain aliphatic alcohols are described in U.S. Pat. No. 5,849,960, U.S. Pat. No. 6,150,222, U.S. Pat. No. 6,222,077, the disclosures of all of which are incorporated herein by reference.

The above-mentioned (non-alkoxylated) alcohol R—OH, from which the hydrocarbyl group R in the above formula (II) originates, may be alkoxylated by reacting with alkylene oxide in the presence of an appropriate alkoxylation catalyst. The alkoxylation catalyst may be potassium hydroxide or sodium hydroxide which is commonly used commercially. Alternatively, a double metal cyanide catalyst may be used, as described in U.S. Pat. No. 6,977,236, the disclosure of which is incorporated herein by reference. Still further, a lanthanum-based or a rare earth metal-based alkoxylation catalyst may be used, as described in U.S. Pat. No. 5,059,719 and U.S. Pat. No. 5,057,627, the disclosures of which are incorporated herein by reference. The alkoxylation reaction temperature may range from 90° C. to 250° C., suitably 120 to 220° C., and super atmospheric pressures may be used if it is desired to maintain the alcohol substantially in the liquid state.

Preferably, the alkoxylation catalyst is a basic catalyst, such as a metal hydroxide, which catalyst contains a Group IA or Group IIA metal ion. Suitably, when the metal ion is a Group IA metal ion, it is a lithium, sodium, potassium or cesium ion, more suitably a sodium or potassium ion, most suitably a potassium ion. Suitably, when the metal ion is a Group IIA metal ion, it is a magnesium, calcium or barium ion. Thus, suitable examples of the alkoxylation catalyst are lithium hydroxide, sodium hydroxide, potassium hydroxide, cesium hydroxide, magnesium hydroxide, calcium hydroxide and barium hydroxide, more suitably sodium hydroxide and potassium hydroxide, most suitably potassium hydroxide. Usually, the amount of such alkoxylation catalyst is of from 0.01 to 5 wt. %, more suitably 0.05 to 1 wt. %, most suitably 0.1 to 0.5 wt. %, based on the total weight of the catalyst, alcohol and alkylene oxide (i.e. the total weight of the final reaction mixture).

The alkoxylation procedure serves to introduce a desired average number of alkylene oxide units per mole of alcohol alkoxylate (that is alkoxylated alcohol), wherein different numbers of alkylene oxide units are distributed over the alcohol alkoxylate molecules. For example, treatment of an alcohol with 7 moles of alkylene oxide per mole of primary alcohol serves to effect the alkoxylation of each alcohol molecule with 7 alkylene oxide groups, although a substantial proportion of the alcohol will have become combined with more than 7 alkylene oxide groups and an approximately equal proportion will have become combined with less than 7. In a typical alkoxylation product mixture, there may also be a minor proportion of unreacted alcohol.

Alkoxylation catalyst that may be contained in the alkoxylated alcohol that is to be contacted with the sulfonic acid in the present invention, originates from a preceding alkoxylation step as described above and usually contains a Group IA or Group IIA metal ion. An advantage of the present invention resides in that no pre-treatment needs to be carried out before contacting the alkoxylated alcohol with the sulfonic acid. For example, in above-mentioned WO199319113, it is disclosed that before contacting the alkoxylated alcohol, which contains residual alkoxylation catalyst, with an acid, first residual alkoxylation catalyst needs to be removed to a certain lower level. In the examples of WO199319113, extractions were carried out to remove excess potassium hydroxide to a level of about 50 ppm before contacting with an acid. Such pre-treatment before carrying out the process of the present invention wherein a sulfonic acid is used as the acid, is advantageously not needed. In the present invention, all of the alkoxylation catalyst from the preceding alkoxylation step can be left in and such alkoxylated alcohol containing alkoxylation catalyst, containing a Group IA or Group IIA metal ion, can then be subjected directly to the process of the present invention wherein said alcohol is contacted with a sulfonic acid As demonstrated in the below Examples, contacting such alkoxylated alcohol with a sulfonic acid resulted in a non-turbid (clear) alkoxylated alcohol containing substantially no solid precipitate, as opposed to other acids which were also tested. Such non-turbid (clear) alkoxylated alcohol may then be advantageously as a starting material in any other process, such as a sulfation process, as further described below.

Accordingly, in the present invention, the alkoxylated alcohol to be contacted with the sulfonic acid may contain a relatively large amount of a Group IA or Group IIA metal ion. In the present invention, said alkoxylated alcohol contains more than 200 parts per million by weight (ppmw) of a Group IA or Group IIA metal ion (based on total weight of the alkoxylated alcohol including other compounds present in the alkoxylated alcohol). Preferably, said amount of the Group IA or Group IIA metal ion in the alkoxylated alcohol is of from 250 ppmw to 5 wt. %, more preferably of from 1,000 ppmw to 1 wt. %, most preferably of from 1,400 to 3,500 ppmw. Preferably, said amount of the Group IA or Group IIA metal ion in the alkoxylated alcohol is at least 250 ppmw, more preferably at least 500 ppmw, more preferably at least 750 ppmw, more preferably at least 1,000 ppmw, more preferably at least 1,200 ppmw, more preferably at least 1,400 ppmw, more preferably at least 1,600 ppmw, more preferably at least 1,800 ppmw, most preferably at least 2,000 ppmw. Further, preferably, said amount of the Group IA or Group IIA metal ion in the alkoxylated alcohol is at most 5 wt. %, more preferably at most 2 wt. %, more preferably at most 1 wt. %, more preferably at most 8,000 ppmw, more preferably at most 6,000 ppmw, more preferably at most 5,000 ppmw, more preferably at most 4,000 ppmw, more preferably at most 3,500 ppmw, more preferably at most 3,000 ppmw, more preferably at most 2,500 ppmw, most preferably at most 2,200 ppmw.

Said Group IA or Group IIA metal ion may originate from the alkoxylation catalyst used in a preceding alkoxylation step as described above. As also described above, when the metal ion as contained in the alkoxylated alcohol is a Group IA metal ion, it is a lithium, sodium, potassium or cesium ion, more suitably a sodium or potassium ion, most suitably a potassium ion. Suitably, when the metal ion is a Group IIA metal ion, it is a magnesium, calcium or barium ion. Preferably, the metal ion as contained in the alkoxylated alcohol is a Group IA metal ion. Further, preferably, said Group IA or Group IIA metal ion originates from the alkoxylation catalyst used in a preceding alkoxylation step. Further, preferably, the alkoxylated alcohol to be contacted with the sulfonic acid contains an alkoxylation catalyst containing said Group IA or Group IIA metal ion, preferably a Group IA metal ion. Further, preferably, the alkoxylation catalyst as contained in such alkoxylated alcohol is selected from lithium hydroxide, sodium hydroxide, potassium hydroxide, cesium hydroxide, magnesium hydroxide, calcium hydroxide and barium hydroxide, more preferably sodium hydroxide and potassium hydroxide, most preferably potassium hydroxide.

The alkoxylated alcohol resulting from contacting an alkoxylated alcohol, which contains more than 200 parts per million by weight of a Group IA or Group IIA metal ion, with the sulfonic acid in accordance with the present invention may be used as a starting material in any process wherein alkoxylated alcohol product is further converted into other valuable chemical products. Advantageously, no further processing step, such as for example removal by filtration of any precipitated salt resulting from the treatment with the sulfonic acid, needs to be carried out, because such precipitates are not formed in the present invention. A specific application where the alkoxylated alcohol product obtained by the process of the present invention may be used is in a process wherein it is sulfated.

Accordingly, the present invention further relates to a process for sulfation of the alkoxylated alcohol resulting from the above-described process of the present invention, wherein the latter alkoxylated alcohol is sulfated by contacting it with a sulfating agent as further described below. Such sulfation process results in a compound of the following formula (III)

[R—O—[R′—O]ₓ—SO₃⁻][Mⁿ⁺]ₒ Formula (III)

wherein R, R′ and x are as described above, M is a counter cation and the product of n and o (n*o) equals 1.

In the above formula (III), n is an integer, which may be 1, 2 or 3, preferably 1 or 2, more preferably 1. Further, o may be any number which ensures that the anionic surfactant is electrically neutral. That is to say, the product of n and o (n*o) should equal 1. o may be a number in the range of from 0.5 to 3.

The counter cation, denoted as Mⁿ⁺ in the above formula (III), may be an organic cation, such as a nitrogen containing cation, for example an ammonium cation which may be unsubstituted or substituted. Further, the counter cation may be a metal cation, such as an alkali metal cation or an alkaline earth metal cation, preferably an alkali metal cation. Preferably, such alkali metal cation is lithium cation, sodium cation or potassium cation.

The alcohol alkoxylate of the above formula (II) may be sulfated using one of a number of sulfating agents including sulfur trioxide, complexes of sulfur trioxide with (Lewis) bases, such as the sulfur trioxide pyridine complex and the sulfur trioxide trimethylamine complex, chlorosulfonic acid, sulfamic acid and oleum. Preferably, the sulfating agent is sulfur trioxide. The sulfation may be carried out at a temperature preferably not above 80° C. The sulfation may be carried out at temperature as low as −20° C., but higher temperatures are more economical. For example, the sulfation may be carried out at a temperature from 20 to 70° C., preferably from 20 to 60° C., and more preferably from 20 to 50° C.

The alcohol alkoxylates may be reacted with a gas mixture which in addition to at least one inert gas contains from 1 to 8 vol. %, relative to the gas mixture, of gaseous sulfur trioxide, preferably from 1.5 to 5 vol. %. Although other inert gases are also suitable, air or nitrogen are preferred, as a rule because of easy availability.

The reaction of the alcohol alkoxylate with the sulfur trioxide containing inert gas may be carried out in falling film reactors. Such reactors utilize a liquid film trickling in a thin layer on a cooled wall which is brought into contact in a continuous current with the gas. Other reactors include stirred tank reactors, which may be employed if the sulfation is carried out using sulfamic acid or a complex of sulfur trioxide and a (Lewis) base, such as the sulfur trioxide pyridine complex or the sulfur trioxide trimethylamine complex, or oleum.

Following sulfation, the liquid reaction mixture may be neutralized using an aqueous alkali metal hydroxide, such as sodium hydroxide or potassium hydroxide, or bases such as ammonium hydroxide, substituted ammonium hydroxide, sodium carbonate or potassium hydrogen carbonate. The neutralization procedure may be carried out over a wide range of temperatures and pressures. For example, the neutralization procedure may be carried out at a temperature from 0° C. to 65° C. and a pressure in the range from 100 to 200 kPa abs. Suitable reactors for this neutralization step comprise a loop reactor and a wiped film evaporator (WFE).

Such sulfates of the above formula (III) may be used as a surfactant, in a various number of applications, including enhanced oil recovery (EOR).

The invention is further illustrated by the following Examples.

### EXAMPLES

In these Examples, the alcohol used was Neodol® 67 which is commercially available at Shell Chemicals. Neodol® 67 is a primary alcohol prepared by hydroformylation of a branched olefin. Said alcohol is of formula R—OH, wherein R is an aliphatic group comprising an alkyl group which is branched, which alcohol contains 1 hydroxyl group (mono-alcohol). Neodol® 67 mainly comprises C16 and C17 alcohols, that is to say alcohols of said formula R—OH wherein R contains 16 and 17 carbon atoms, respectively (C16: 31 wt. %; C17: 54 wt. %).

Said Neodol® 67 was propoxylated using propylene oxide in such an amount that the average number of propylene oxide units in the resulting Neodol® 67 propoxylate was 6.8. The alkoxylation catalyst used was potassium hydroxide (KOH).

The alkoxylation procedure was as follows. Neodol® 67 (molecular weight: 251 g/mole) in an amount of 700 g (2.8 moles) and a composition, comprising 85 wt. % of KOH the remainder being water, were mixed. The mixture was heated to 120° C. and a nitrogen sparge was applied to remove water. The mixture was then transferred to a propoxylation reactor. Then the propylene oxide was added to the mixture at a rate varying between 1 and 5 grams per minute (autogeneous, via pressure control). The total amount of propylene oxide (molecular weight=58.1 g/mole) added was 1133.8 g (19.5 moles). The reaction temperature was 120° C. The amount of the added KOH catalyst containing composition was 0.35 wt. % based on the total weight of the reaction mixture after all propylene oxide had been added. The amount of added KOH catalyst as such (that is to say excluding the water) was therefore 0.30 wt. %. Consequently, the amount of added K (potassium) as such was 0.21 wt. %, that is to say about 2,100 parts per million by weight (ppmw). After all propylene oxide had been added the mixture was left to completion of the alkoxylation reaction overnight. Upon subsequent cooling of the reaction mixture to 50° C., either no acid was added or an acid was added. In the table below, the various acids tested are mentioned. During neutralization, the temperature was maintained at 50° C. to ensure that all acids were liquid (lauric acid is solid at room temperature). The amount of acid added was equimolar to the amount of KOH catalyst.

| | Appearance of reaction |
|---|---|
| Acid added | mixture after acid addition |
| none | clear |
| acetic acid | haze |
| oleic acid | haze |
| lauric acid | haze |
| para-alkylbenzene sulfonic | clear |
| acid(1) | |

(1)This acid is commercially available at Acros Organics: “Product 32590 Dodecylbenzene sulfonic acid, mixture of C10-C13 isomers” (CAS 85536-14-17), which is a para-alkylbenzene sulfonic acid wherein the alkyl group is mostly linear and wherein the carbon numbers for the alkyl group are distributed over 10, 11, 12 and 13 carbon atoms.

In the table above, the appearance of the reaction mixture is described, either of the non-neutralized reaction mixture or of the reaction mixture after addition of an acid. From that it appears that when neutralizing the KOH catalyst in the reaction mixture using para-dodecylbenzene sulfonic acid, which is a sulfonic acid in accordance with the present invention, advantageously, the reaction mixture remained clear and no solids were produced. On the other hand, when using acids other than sulfonic acids, such as acetic acid, oleic acid and lauric acid, during the neutralization a haze was developed in the reaction mixture caused by potassium salt precipitation.

Further, the following alcohols were propoxylated by applying the alkoxylation procedure as described above: Neodol® 67, 2-ethyl hexanol and 1-hexadecanol. Upon cooling of the reaction mixture, either no acid was added or the para-alkylbenzene sulfonic acid as described above (hereinafter “DDBSA”) was added for neutralization. Then the turbidity of the reaction mixture was measured. The turbidity measurements were made using a Beckman Probe Colorimeter Model PC950, employing reflecting probe with a path length of 1 cm from the light source to the mirror. This probe measures % transmittance from visible light source centered on 520 nm. The results of these measurements are shown in the table below.

% Transmittance

| | Not | Neutralized by |
|---|---|---|
| | neutralized | DDBSA |
| Neodol ® 67 propoxylate | 84 | 92 |
| 2-ethyl hexanol propoxylate | 40 | 3.3 |
| 1-hexadecanol propoxylate | 83 | 74 |

From the above results it appears that neutralization by DDBSA advantageously results in more transmittance (less turbidity) for the propoxylate of Neodol® 67 (92%) as compared to the propoxylates of 2-ethyl hexanol and 1-hexadecanol (3.3% and 74%, respectively). Furthermore, it appears that using DDBSA for the propoxylates of 2-ethyl hexanol and 1-hexadecanol actually results in a decrease of transmittance (decrease by 36.7% and 9%, respectively), as compared to the unneutralized case, whereas for Neodol® 67 this advantageously results in an increase of transmittance (increase by 8%).

1. A process for preparing alkoxylated alcohols, wherein an alkoxylated alcohol which contains more than 200 parts per million by weight of a Group IA or Group IIA metal ion is contacted with a sulfonic acid.

2. A process according to claim 1, wherein the alkoxylated alcohol contains more than 200 parts per million by weight of a Group IA metal ion which is a lithium, sodium, potassium or cesium ion.

3. A process according to claim 1, wherein the alkoxylated alcohol contains an alkoxylation catalyst containing said Group IA or Group IIA metal ion.

4. A process according to claim 3, wherein the alkoxylation catalyst is selected from lithium hydroxide, sodium hydroxide, potassium hydroxide, cesium hydroxide, magnesium hydroxide, calcium hydroxide and barium hydroxide.

5. A process according to claim 1, wherein the alkoxylated alcohol contains the Group IA or Group IIA metal ion in an amount of from 250 parts per million by weight to 5 wt. %.

6. A process according to claim 1, wherein the sulfonic acid is of the formula (I)
R—S(═O)₂—OH Formula (I)
wherein R is a hydrocarbyl group.

7. A process according to claim 6, wherein R is an aromatic group.

8. A process according to claim 7, wherein R is a phenyl group.

9. A process according to claim 8, wherein the phenyl group is substituted by 1 or more.

10. A process according to claim 1, wherein the alkoxylated alcohol is of the formula (II)
R—O—[R′—O]ₓ—H Formula (II)
wherein R is a hydrocarbyl group, R′—O is an alkylene oxide group and x is the number of alkylene oxide groups R′—O which is at least 0.5.

11. A process according to claim 1, wherein after contacting the alkoxylated alcohol with the sulfonic acid, the alkoxylated alcohol is sulfated by contacting the alkoxylated alcohol with a sulfating agent.

12. A process according to claim 11, wherein the sulfating agent is selected from sulfur trioxide, complexes of sulfur trioxide with bases, chlorosulfonic acid, sulfamic acid and oleum.
